(19) United States
(12) Patent Application Publication
(10) Pub. No.: US 2016/0376541 A1
(43) Pub. Date: Dec. 29, 2016

(54) PROCESS FOR SAPONIN ENHANCED AUTOLOYSIS OF YEAST

(71) Applicant: Minn-Dak Farmers Cooperative, Wahpeton, ND (US)

(72) Inventors: Richard Ames, Wahpeton, ND (US); Jeffrey Sobolik, Reynolds, ND (US)

(21) Appl. No.: 15/190,407

(22) Filed: Jun. 23, 2016

Related U.S. Application Data
(60) Provisional application No. 62/183,207, filed on Jun. 23, 2015.

(51) Int. Cl. C12N 1/06; C12P 1/02

(52) U.S. Cl. CPC C12N 1/063; C12P 1/02

(57) ABSTRACT

Process for enhancing production rates/production of yeast cell wall products and yeast extracts by adding saponin or a saponin containing ingredient to yeast cultures during fermentation or to yeast cream prior to autolysis. In the context of a sugar beet processing facility, saponin, which is contained within the sugar refining process streams during sucrose production, is readily available and can be introduced to the yeast cultures or yeast cream as either a saponin extract or as dried and shredded sugar beet leaves, without requiring any additional sourcing or acquisition costs. Activity between the saponin and yeast/yeast cream results in the formation of saponin fermentation products.

### RELATED APPLICATION

The present application claims the benefit of U.S. Provisional Application No. 61/183,207 filed Jun. 23, 2015, which is hereby incorporated herein in its entirety by reference.

### FIELD OF THE INVENTION

The present invention relates generally to processes for the production of yeast products. More specifically, the present invention is directed to processes incorporating saponin to intentionally disrupt and damage a yeast cell wall so as to selectively enhance production of yeast extract flavorings, yeast cell wall products and saponin fermentation products.

### BACKGROUND OF THE INVENTION

Baker's yeast (Saccharomyces cerevisiae) is a species of yeast that has been used for bread making, winemaking and brewing for thousands of years. Generally, baker's yeast can be grown using sugars such as, for example, sucrose, fructose, glucose, maltose and trehalose and as such, can be a viable product for processes in which these sugars are readily available. One specific application in which such sugars are readily available for consumption is in a sugar beet processing facility in which the primary and secondary products of beet sugar (sucrose) and molasses, respectively, are produced and readily available to feed the baker's yeast.

When being supplied to traditional yeast consumers such as for the production of leavened dough products or fermented beverages, a satisfactory baker's yeast is in a compressed or cream form. In these compressed or cream forms, the cell wall of the baker's yeast is strong enough that the yeast cell walls remain stable so as to tolerate heat, cold and osmotic stress.

While the production of compressed or cream baker's yeast is advantageous with traditional fermentation uses, there may be other yeast applications in which having a strong cell wall may be disadvantageous to process results. As such, it would be beneficial to develop ways for selectively producing baker's yeast having a weakened or less robust cell wall for processes in which cell wall products or yeast extract flavoring are desirable.

### SUMMARY OF THE INVENTION

The processes of the present invention address the desire of producing yeast cells with a weakened or less robust cell wall through the selective introduction of elevated levels of saponin. Saponin, a fungicide which is found in many plants, is a group of amphipathic glycosides that are known for their flocculent properties in aqueous solution. When added during fermentation or to yeast cream, metabolic activity between the saponin and the yeast/yeast cream results in, for example, increased RNA and Free Amino Nitrogen (FAN) release during yeast autolysis, thereby indicating damage and/or weakening of the cell wall membrane. By adding saponin to yeast cream, the production and/or production rate of cell wall components and yeast extracts can be increased. In the context of a sugar beet processing facility, saponin, which is contained within the processing streams during sucrose production, is readily available and can be introduced during fermentation or to the cream yeast without requiring any additional sourcing or acquisition costs. In addition to the production of the cell wall components and yeast extracts, the activity between the saponin and yeast/yeast cream results in the formation of saponin metabolites.

In one aspect, the present invention is directed to a process for enhancing the production of yeast cell wall components and yeast extracts. Generally, the process can comprise adding saponin during fermentation or to yeast cream prior to yeast autolysis. As a result of metabolic interaction between the yeast and saponin in the fermenter or yeast cream, the amount and/or rate of RNA release during yeast cell autolysis can be increased. An increase in RNA release indicates disruption and/or weakening of the yeast cell wall membrane. An increase in the amount or rate of RNA release corresponds with an increase in production of yeast autolysis products. Representative manufacturers that produce, for example, protein hydrolysate, food flavoring ingredients such as 5′ nucleotide 10% I & G, basic yeast extracts and beta glucan from yeast cell wall components and extracts can see the production of these yeast autolysis products enhanced. In addition to the production of the yeast cell wall components and yeast extracts, the activity between the saponin and yeast/yeast cream results in the formation of saponin metabolites.

In another aspect, the present invention is a process for using a saponin product that is isolated during agricultural processing, for example, sugar beet processing, to selectively produce and/or increase the amounts and production rates of yeast cell wall components and yeast extracts. The process can utilize isolated saponin extracts or dried plant materials containing saponins. The saponin may either be processed or naturally occurring. The process can comprise of adding saponin during fermentation or to yeast cream prior to yeast autolysis. In addition to the production of the yeast cell wall components and yeast extracts, the activity between the saponin and yeast/yeast cream results in the formation of saponin metabolites.

In yet another aspect, the present invention can comprise a process for intentionally growing yeast having a damaged and/or weakened yeast cell wall membrane. The process can comprise adding saponin during fermentation or to yeast cream prior to yeast autolysis. The process can further comprise increasing the rate of production and/or yield of yeast cell wall components and yeast extracts. The process can comprise carrying out the steps under either anaerobic or aerobic conditions. The process can further comprise the formation of saponin metabolites.

In another aspect, the present invention can comprise a method for increasing a production rate and/or yield of yeast cell wall components and yeast extracts through the introduction of saponin during fermentation or to a yeast cream prior to yeast autolysis. Yeast species that could be targeted for saponin treatment can include, for example, strains of saccharomyces cerevisiae (baker's and brewer's yeast), kluyveromyces fragilis, and candida strains, such as candida utilis, and combinations thereof, saccharomyces delbruekii, saccharomyces rosei, saccharomyces microellipsodes, saccharomyces carlsbergensis, schizosaccharomyces pombe, kluyveromyces lactis, kluyveromyces polysporus, candida albicans, candida cloacae, candida tropicalis, candida guilliermondii, hansenula wingei, hansenula arni, hansenula henricii, hansenula americana and combinations thereof. In addition, metabolic activity between the saponin and yeast/yeast cream results in the formation of saponin metabolites.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follow more particularly exemplify these embodiments.

### BRIEF DESCRIPTION OF THE DRAWINGS

The invention can be completely understood in consideration of the following detailed description of various embodiments of the invention in connection with the accompanying drawings, in which:

FIG. 1 illustrates the Optical Density (OD) over time for measuring the saponin effects on the growth of yeast cultures during fermentation.

FIG. 2 demonstrates the enhanced RNA release for a sample in which saponin was added to yeast cream at a fermentation of 30° C. for 2 hours as compared to the control.

FIG. 3 illustrates the concentration ratio of RNA corresponding to the samples illustrated in FIG. 2.

FIG. 4 illustrates saponin enhanced autolysis of baker's yeast through the measurement of free amino nitrogen.

FIG. 5 illustrates saponin enhanced autolysis at 50° C. through the measurement of free amino nitrogen at 24 hours.

FIG. 6 illustrates saponin enhanced autolysis at 50° C. through the measurement of free amino nitrogen at 48 hours.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

Processes according to representative embodiments of the present invention can be utilized to selectively increase the production rate and/or yield of yeast cell wall components and yeast extracts. Generally, the process involves the selective addition of saponin during fermentation (either batch fermentation, fed batch fermentation or continuous fermentation) or to a yeast cream prior to yeast autolysis whereby metabolic activity between the yeast and the saponin results in a damaged and/or weakened yeast cell wall membrane. Damaged/weakened yeast cell wall membranes are generally indicated by the increased presence of RNA in the resulting autolysates. An increased presence of RNA in the resulting autolysates indicates an increased presence of yeast autolysis products including cell wall, flavoring and extract products. Representative yeast species that can be targeted for the saponin treatment of the present invention can include, for example, strains of saccharomyces cerevisiae (baker's and brewer's yeast), kluyveromyces fragilis, and candida strains, such as candida utilis, and combinations thereof, saccharomyces delbruekii, saccharomyces rosei, saccharomyces microellipsodes, saccharomyces carlsbergensis, schizosaccharomyces pombe, kluyveromyces lactis, kluyveromyces polysporus, candida albicans, candida cloacae, candida tropicalis, candida guilliermondii, hansenula wingei, hansenula arni, hansenula henricii, hansenula americana and combinations thereof. In addition to the production of the yeast cell wall products, the activity between the saponin and yeast/yeast cream results in the formation of saponin metabolites.

Saponin is an amphipathic glycoside that is frequently found within various plant species including sugar beets and possesses fungicidal properties. When using beet sugar in various applications, the presence of saponin has been found to be disadvantageous due to its floc properties. For example, when saponin is present within beet sugar used in the beverage industry that produces low pH carbonated soft drinks, as an example, the resulting beverage can suffer from quality problems (cloudiness) due to flocculation.

Minn-Dak Farmers Cooperative of Wahpeton, North Dakota is a sugar beet processor that processes sugar beets to recover sucrose. In addition to beet sugar, Minn-Dak also has a yeast plant that utilizes a byproduct from the refining process, molasses, to produce baker's yeast. In producing baker's yeast, Minn-Dak has identified certain conditions in which the production of compressed yeast is compromised resulting in what has been traditionally considered an unacceptable “gummy” yeast product. The gummy consistency is considered poor quality for traditional baker's yeast consumers such as commercial bakeries preparing dough products and breweries making fermented beverages. However, the resulting gummy consistency of the yeast product is indicative of cell wall membrane damage which can be advantageous to other users of yeast cell wall products and yeast extracts. As such, Minn-Dak has discovered a repeatable process to intentionally enhance the production of yeast cell wall products and yeast extracts through the selective introduction of saponin to yeast cultures during fermentation or to yeast cream prior to yeast autolysis. In addition, the presence of saponin in the processing streams of Minn-Dak's beet sugar process provides an inexpensive and readily available mechanism for selectively enhancing the production and/or rate of production of yeast cell wall products and yeast extracts in the existing yeast production facility.

### Saponin Addition During Fermentation

In order to produce baker's yeast, a carbon-based energy source is necessary for yeast propagation. Traditionally, the fermentation process has been performed with the intention of growing yeast that ultimately assumes a compressed form suitable for use in the baking industry. In the following examples, saponin is intentionally added, either as a component of the energy source or as a supplement to the energy source, to yeast cultures during a fermentation period to determine the impact of saponin on yeast cell development.

Throughout the following examples, the yeast used in all experiments was primary grown baker's yeast obtained from Minn-Dak Yeast Company production fermentation tanks. All yeast samples were tested for gassing and heat shock stability to verify the health and vitality of the yeast sample. Only high quality, stable yeast was used for the experiments. Examples 1 and 2 were performed within a laboratory flask while a 14-liter, New Brunswick Scientific Company Microferm Fermentor was used as an autolysis reactor for Examples 3 and 4. The Microferm had temperature and pH control with an agitator rotating at 400 rpm with 3, 6-bladed paddle wheel style impellers. Process variables including mixing, pH and temperature were controlled throughout the examples.

### EXAMPLE 1

Three samples were prepared in which yeast cultures were propagated in a flask. The energy source for each sample was sugar beet molasses, comprising mainly of sucrose, glucose, and fructose. Generally, the sugar beet molasses is available as a byproduct of sugar beet processing.

In sample 1, the sugar beet molasses was supplied directly to the flask with no pretreatment/filtering. In sample 2, the sugar beet molasses was filtered prior to being added to the flask to remove any saponin prior to exposure to the yeast culture. In sample 3, the sugar beet molasses had a controlled amount of saponin added prior to fermentation. Over the course of fermentation, the Optical Density (OD) was routinely measured for each sample, wherein higher OD measurements correspond with increased yeast cell growth. Correspondingly, lower OD measurements indicate reduced yeast cell growth.

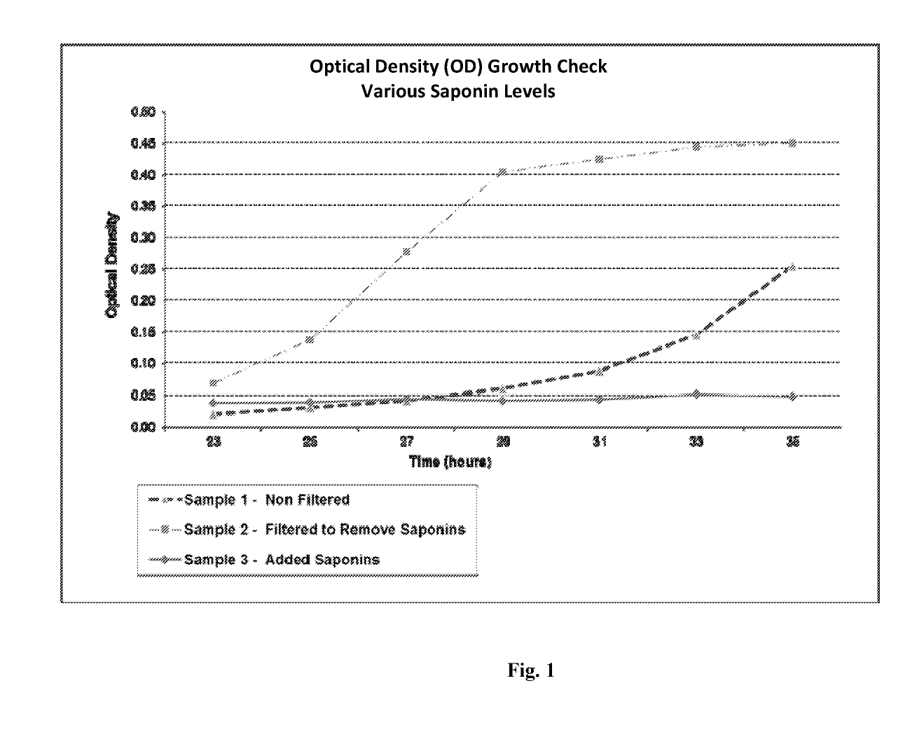

The OD results for samples 1, 2 and 3 are summarized in FIG. 1. Generally, the results show that the removal of saponin prior to fermentation (sample 2) results in the highest yeast cell growth, while saponin enriched molasses (sample 3) significantly impedes yeast cell growth. The control sample (sample 1) appeared to indicate the presence of lower levels of saponin (as compared to sample 3), which would be expected in sugar beet molasses that experienced no filtering prior to fermentation.

### Saponin Addition to Yeast Cream

When saponin is added to yeast cream, metabolic activity between the saponin and yeast cream results in damage to and/or weakening of the yeast cell wall membrane. The resulting damage/weakening of the yeast cell wall membrane can be quantified by measuring the amount of RNA released during yeast autolysis with a spectrophotometer set at a wavelength of 260 nm. The increase in RNA release due to saponin addition is demonstrated in the experimental example below.

### EXAMPLE 2

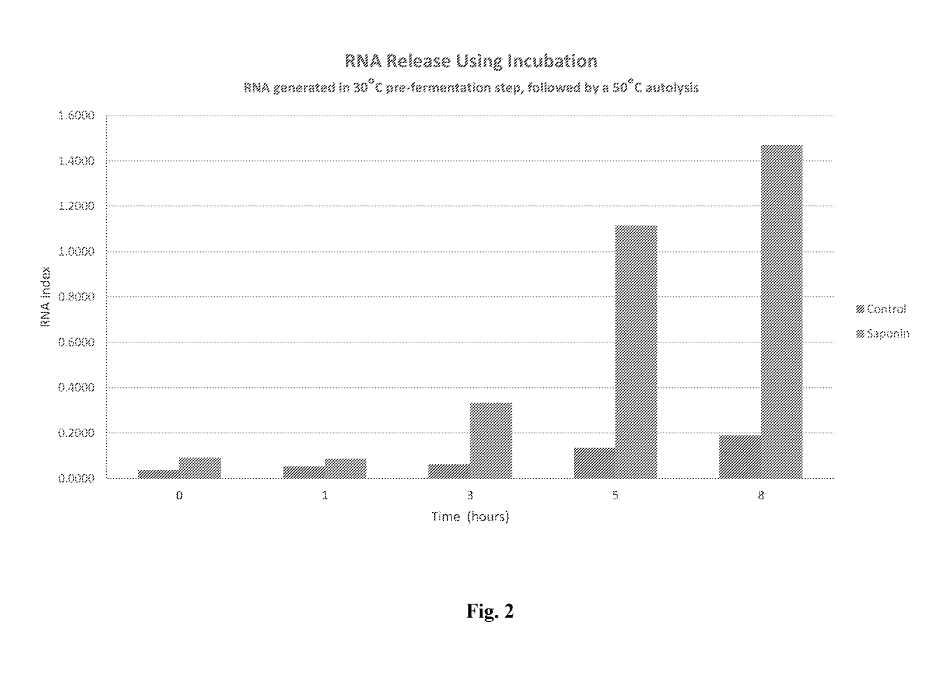

Two samples of yeast cream were prepared and incubated simultaneously. The first sample was a control which contained only yeast cream. The second sample contained yeast cream identical to the control but had a known amount of saponin extract added to the mixture. The two samples were incubated under identical conditions at a temperature of 30° C. for a period of two hours in a temperature controlled water bath to initiate metabolic activity between the saponin and yeast cream. After two hours, the two samples were then heated to 50° C. in less than 10 minutes after which time they stayed at that temperature for a period of six hours to accomplish yeast autolysis. During autolysis, each of the two samples were analyzed periodically with a spectrophotometer at 260 nm to measure RNA release and the absorbance indexes are shown in FIG. 2.

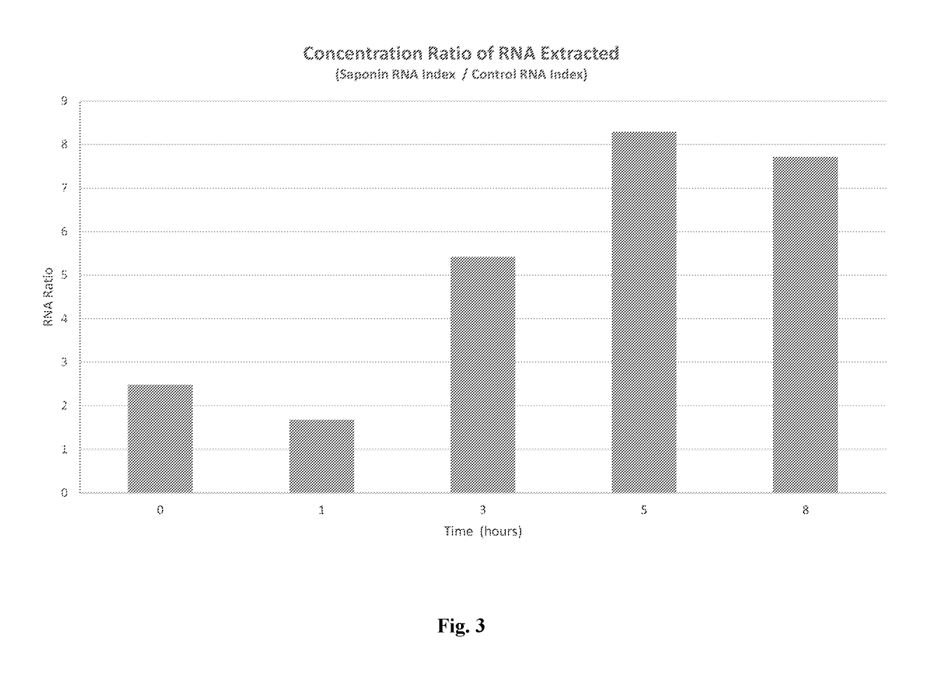

As seen in FIG. 2, the saponin containing sample had a significantly higher RNA index as compared to the control for shared time intervals. If the ratio of the RNA concentrations of the two test mixtures are plotted over time, one can see in FIG. 3 that the saponin enhanced autolysis generated two to eight times more RNA than the control during the eight hour test period.

As indicated in the testing, the introduction of saponin and/or saponin containing by-products from sugar beet processing results in higher levels of RNA being released during yeast autolysis. The presence of higher levels of RNA is an indication of yeast cell wall damage/weakening and is beneficial when the desired products are yeast cell wall products and yeast extracts. By selectively controlling the addition of saponin during yeast fermentation or prior to autolysis, a yeast end product, either in compressed form with a robust cell wall for commercial bakeries and breweries or gummy yeast for yeast cell wall products and yeast extracts, can be selectively produced. More specifically, the production of yeast cell wall products and yeast extracts, for example, protein hydrolysate, food flavoring ingredients such as 5′ nucleotide 10% I & G, basic yeast extracts and beta glucan can be increased through the introduction of saponin to yeast cream prior to autolysis.

### Saponin Enhanced Autolysis

In the following Examples 3 and 4, a saponin extract (extracted during sugar beet processing) was utilized in the yeast autolysis testing. The saponin extract consisted of:

| Saponin Extract | Weight % |
|---|---|
| Sugar | ≈65% |
| Protein | ≈5% |
| Saponin | ≈30% |

### EXAMPLE 3

Three samples of baker's yeast were prepared. The first sample was a control sample of high quality baker's yeast with no saponin added. The second sample contained high quality baker's yeast with 70 g of saponin extract added as a detergent under conditions in which little to no fermentation activity occurs between the yeast and the saponin. The third sample contained high quality baker's yeast with 70 g of saponin extract added as fermentation feed at 30° C. for 2 hours prior to the autolysis step. The 3 samples of baker's yeast were placed in an autolysis reactor at 45° C. and at a pH of 5.45-5.55. Samples were drawn from the reactor at 24 and 48 hours into the autolysis. 50 ml aliquots of the samples were spun in a centrifuge at 3300 rpm for 8 minutes. The samples had a yeast cell wall pellet on the bottom of the tube and a light phase extract liquid on the top portion. The light phase extract was filtered through a diatomaceous earth filter and analyzed for free amino nitrogen concentration (FAN). FAN was used as an indication of autolytic activity. Higher concentrations of FAN indicated a greater release of yeast cell proteins and proteolytic enzymes.

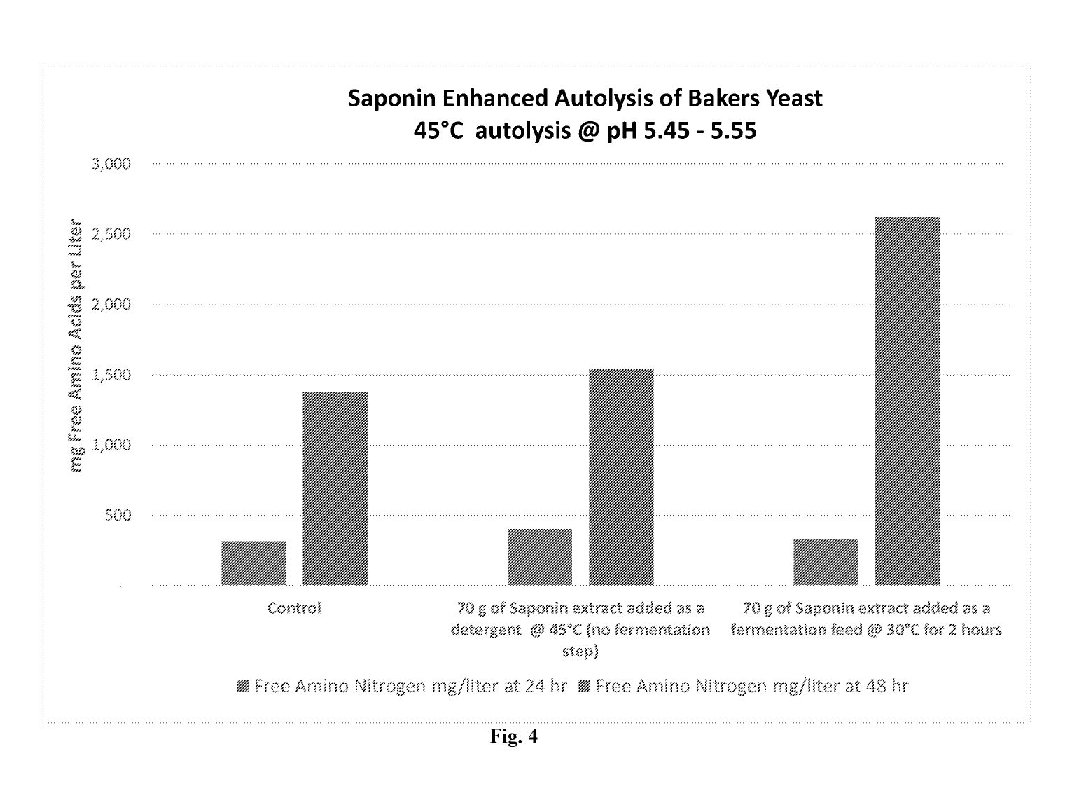

As seen in FIG. 4, all three samples had similar FAN concentrations at 24 hours into autolysis. Sample 2, without a pre-autolysis fermentation step, had roughly the same FAN concentration as the control. However, at 48 hours, sample 3 had a 91% increase in FAN over the control. The reaction in sample 2 was carried out under conditions in which saponin is a non-biologically active detergent, without a fermentation step. The results of sample 3 indicate that the addition of saponin during yeast fementation under biologically active conditions has the effect of increasing autolytic activity. This indicates that the introduction of saponin during a fermentation step ultimately accelerates autolysis of yeast. The mechanism for these results is believed to be the interaction of saponin glycosides and the yeast cell wall under conditions that promote fermentation activity.

### EXAMPLE 4

Four samples of baker's yeast were prepared. The first sample was a control sample of high quality baker's yeast with no added saponin. The second sample contained high quality baker's yeast with 35 g of saponin extract containing approximately 10 g of pure saponin added as a fermentation feed at 30° C. 2 hours prior to autolysis. The third sample contained high quality baker's yeast with 202 g of dried and shredded sugar beet leaves containing approximately 10 g of pure saponin added as a fermentation feed at 30° C. 2 hours prior to autolysis. The fourth sample included high quality baker's yeast with 70 g of saponin extract containing approximately 20 g of pure saponin added as a fermentation feed at 30° C. for 2 hours prior to autolysis. The samples were placed in an autolysis reactor at 50° C. and a pH of 5.45-5.55. The samples were drawn from the reactor at 24 and 48 hours into the autolysis. 50 ml aliquots of the samples were spun in a centrifuge at 3300 rpm for 8 minutes. The centrifuged samples had a yeast cell wall pellet on the bottom of the tube and a light phase extract liquid on the top portion. The light phase extract was filtered through a diatomaceous earth filter and analyzed for FAN concentration.

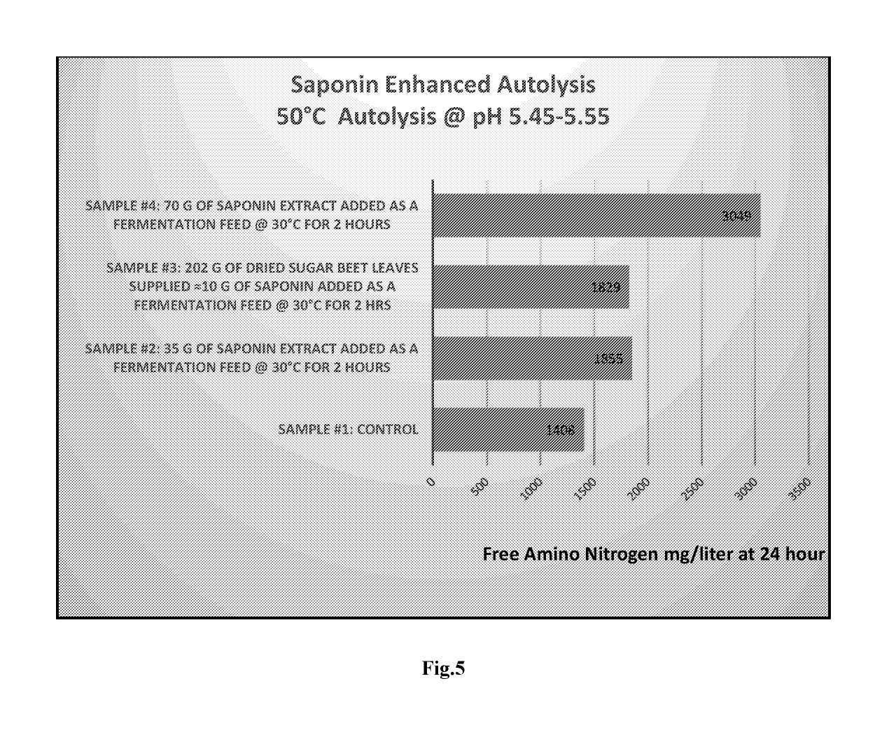

As seen in FIG. 5, the 50° C. autolysis series showed significantly more FAN in the yeast extract at 24 hours into the autolysis than the 45° C. series across all autolysis conditions. This indicates that elevated temperatures have a significant impact on autolysis of fungal cell walls. At 24 hours, Samples 2 and 3 supplied about the same amount of actual saponin to the autolysis experiment and the FAN concentration increase over the control was very similar at 30% and 32% respectively. These results indicate that equivalent amounts of saponin, supplied either in the form of a processed extract or in its natural state (dried and shredded sugar beet leaves), has the equivalent effect of increasing autolytic activity regardless of the saponin source. Sample 4 possessed double the saponin concentration as compared to samples 2 and 3, and the FAN concentration of sample 4 was increased 117% over the control (sample 1), indicating that higher saponin concentrations increase autolysis rates.

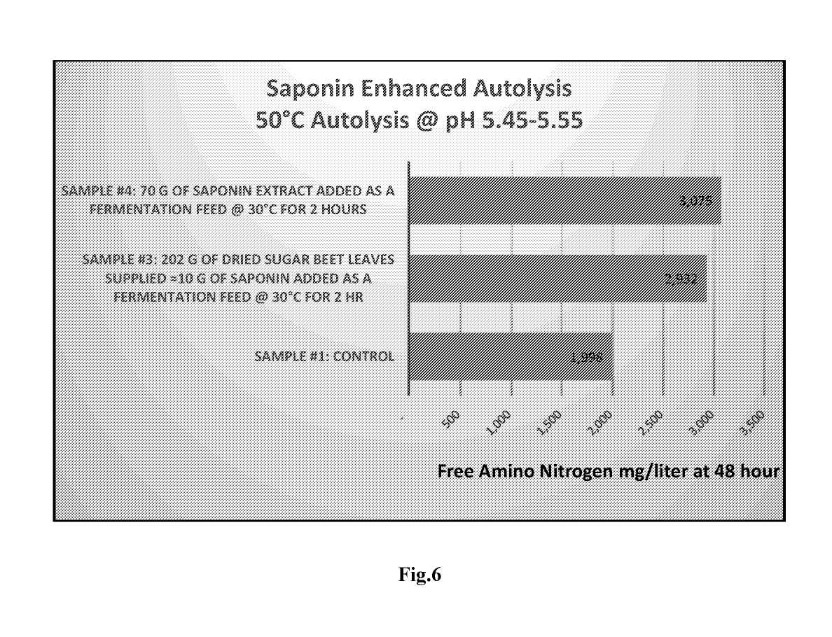

As seen in FIG. 6, the 50° C. samples had more similar FAN concentrations over the sample range compared to the 45° C. experiments after 48 hours of autolysis. This is due to the enhanced release of yeast cell contents into the extract solution at higher temperatures with and without saponin added. FIG. 6 also indicates that sample3, with roughly half the amount of saponin content of sample 4, had similar amounts of autolytic activity as that of sample 4 after 48 hours. Sample 2 was not tested at 48 hours, so no data is presented for sample 2 in FIG. 6. These results indicate that increased saponin concentrations increase the rate of autolytic activity of yeast. Referring to FIG. 5, sample 4 was essentially 99% complete at 24 hours, while samples 2 and 3 were only 62% complete at 24 hours. However, after 48 hours, sample 3 had experienced essentially the same amount of autolytic activity as sample 4.

As indicated by the testing, the addition of saponin to the autolysis process of baker's yeast proved to effectively increase autolysis rates when there was a pre-autolysis fermentation step. When using either the processed saponin extract or the dried and shredded sugar beet leaves, similar results were obtained for samples containing the same amounts of saponin regardless of saponin source. While these experiments were carried out under anaerobic conditions, similar results are expected under aerobic processing conditions. Commercial applications for this process can be traditional yeast autolysis, yeast extract production, yeast cell wall and cell wall product production, and saponin fermentation products among others. Other saponin sources such as, for example, as a product or byproduct of other agricultural sources such as soybeans, peanuts, various bean species, oats, asparagus, spinach, alfalfa and various tree species can have the same effect. Due to the presence of different and unique saponins in these various agricultural products, it is expected that the use of different agricultural sources for the saponin will allow for the production of a variety of different yeast autolysis and saponin fermentation products. Regardless of the saponin source, the fungal or yeast strains exposed to saponins during a fermentation step will respond by having weakened cell walls and increased rates and amounts of autolysis products that will vary based upon the processing conditions and the saponin source.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents.

1. A method for enhancing the autolysis of yeast comprising:
adding saponin to a yeast culture during fermentation;
fermenting the yeast in the presence of saponin; and
carrying out yeast autolysis in the presence of saponin.

2. A method for enhancing the autolysis of yeast according to claim 1, further comprising:
obtaining the saponin as an agricultural processing product.

3. The method of claim 2, wherein the step of obtaining the saponin further comprising:
obtaining the saponin as a sugar beet product.

4. The method of claim 3, wherein the step of obtaining the saponin further comprising:
supplying the saponin to the yeast in a molasses feedstream.

5. The method of claim 4, further comprising:
adding an amount of additional saponin to the molasses feedstream.

6. The method of claim 5, wherein the additional saponin comprises a saponin extract.

7. The method of claim 5, wherein the saponin comprises shredded and dried sugar beet leaves.

8. The method of claim 1, wherein the saponin is a plant derived saponin.

9. The method of claim 1, wherein the yeast culture is selected from the group consisting essentially of: saccharomyces cerevisiae (baker's and brewer's yeast), kluyveromyces fragilis, and candida strains, such as candida utilis, and combinations thereof, saccharomyces delbruekii, saccharomyces rosei, saccharomyces microellipsodes, saccharomyces carlsbergensis, schizosaccharomyces pombe, kluyveromyces lactis, kluyveromyces polysporus, candida albicans, candida cloacae, candida tropicalis, candida guilliermondii, hansenula wingei, hansenula arni, hansenula henricii, hansenula americana and combinations thereof.

10. The method of claim 1, wherein the fermentation step comprises batch fermentation, fed batch fermentation, or continuous fermentation.

11. The method of claim 1, further comprising the step of:
elevating temperature during yeast autolysis.

12. The method of claim 1, further comprising:
monitoring autolytic activity by measuring RNA concentration during yeast autolysis.

13. The method of claim 1, further comprising:
monitoring Free Amino Nitrogen (FAN) concentration during yeast autolysis.

14. The method of claim 1, wherein the yeast autolysis produces elevated levels of yeast cell wall products and yeast extracts.

15. The method of claim 14, wherein the production time of the elevated levels of yeast cell wall product and yeast extracts is reduced through the addition of increased levels of saponin.

16. The method of claim 1, wherein yeast autolysis results in the conversion of the saponin into saponin fermentation products.

17. The method of claim 1, wherein the yeast culture comprises a yeast cream.

18. The method of claim 1, wherein the fermenting step is carried out within an autolysis reactor.

19. A gummy yeast product including a damaged or otherwise weakened yeast cell wall membrane produced according to the method of claim 1.

20. A saponin fermentation product produced according to the method of claim 1.
